(19) United States
(12) Patent Application Publication
(10) Pub. No.: US 2019/0329257 A1
(43) Pub. Date: Oct. 31, 2019

(54) SYSTEM FOR DIAGNOSING MALE INFERTILITY

(71) Applicant: Young Jae KIM, Seongnam-si (KR)

(72) Inventor: Young Jae KIM, Seongnam-si (KR)

(21) Appl. No.: 16/308,919

(22) PCT Filed: Jun. 16, 2017
(86) PCT No.: PCT/KR2017/006318

§ 371 Date: Dec. 11, 2018

(30) Foreign Application Priority Data
Jun. 17, 2016 (KR) 10-2016-0075779

(51) Int. Cl. B01L 3/00; A61B 10/00; G01N 15/14

(52) U.S. Cl. CPC B01L 3/502761; A61B 10/0058; B01L 3/502715; B01L 2300/0861; G01N 15/1436; B01L 2300/0663; B01L 2200/0668; G01N 15/1484

(57) ABSTRACT

Disclosed herein is a system for diagnosing male infertility including a microfluidic chip, wherein the microfluidic chip includes a first chamber cover having an injection port allowing a medium and a sperm sample to be injected therethrough, and a chamber coupled to a lower portion of the first chamber cover and provided with a plurality of microfluidic channels, the plurality of microfluidic channels being arranged to converge in a direction in which spermatozoa progress.

### TECHNICAL FIELD

The present invention relates to a male infertility diagnosis system for diagnosing male infertility using forward progression of spermatozoa.

### BACKGROUND ART

Recently, male infertility has been increasing due to various causes such as environmental pollution and excessive stress. It is generally known that causes of male infertility include decreased sperm count, and decreased mobility and abnormal shape of spermatozoa.

In order to diagnose male infertility, a sperm sample is taken from a male and is observed under a microscope. The spermatozoa seen in the field of the microscope and the spermatozoa that move with forward progression are respectively counted, and the ratio therebetween is measured. When the proportion of spermatozoa that move with forward progression is higher than a reference value, it is determined that the sperm are normal. Various kinds of systems and equipment for such diagnosis have been developed and utilized.

Currently, most male infertility diagnoses are performed at hospitals, medical centers, or infertility clinics. However, due to personal privacy and inconvenience caused in the process of diagnosis, there is increasing demand for portable devices for diagnosis of male infertility.

However, portable male infertility diagnostic devices proposed to date have low accuracy or user convenience or are comparatively expensive, thereby providing low user accessibility.

In addition, it is difficult to use the conventional male infertility diagnostic devices in conjunction with a smart device such as a smartphone since sperm are observed through color classification by chemical action or a microscope.

Related technologies include Korean Patent Application Publication No. 10-2009-0132267 (Publication date: Dec. 30, 2009) entitled “Diagnostic Devices Having Microchannel.”

### DISCLOSURE

### Technical Problem

Therefore, the present invention has been made in view of the above problems, and it is one object of the present invention to provide a male infertility diagnosis system which can be manufactured at low cost using a relatively simple structure and which improves the accuracy of infertility diagnosis by suggesting a new structure including a feedback channel or an air collector.

It is another object of the present invention to provide a male infertility diagnosis system which is equipped with a sperm detector such as a CCD sensor and thus can acquire infertility-related information by monitoring the sperm, and easily transmit the infertility-related information to a smart device through wireless connection, such as Bluetooth connection or Wi-Fi connection, or wired connection such as USB cable connection to facilitate storage of the infertility-related information and observation of changes through graphs or the like.

The objects to be achieved by the present invention are not limited to the above-mentioned object(s), and other object(s) not mentioned can be clearly understood by those skilled in the art from the following description.

### Technical Solution

In accordance with one aspect of the present invention, provided is a system for diagnosing male infertility including a microfluidic chip, wherein the microfluidic chip includes a first chamber cover having an injection port allowing a medium and a sperm sample to be injected therethrough, and a chamber coupled to a lower portion of the first chamber cover and provided with a plurality of microfluidic channels, the plurality of microfluidic channels being arranged to converge in a direction in which spermatozoa progress.

The system further includes a second chamber cover coupled to a lower portion of the chamber to form a base surface of the microfluidic channels.

The system further includes a sperm detector arranged under the second chamber cover to monitor the spermatozoa in the chamber through diffraction of light emitted from a light source.

The microfluidic channels have a width reduced from one end of the microfluidic channels to the other end of the microfluidic channels.

The injection port is opened to cover at least a part of each of the microfluidic channels.

In addition, the injection port is provided with a concave portion curved toward one end of the microfluidic channels.

Further, the concave portion is formed to extend over the microfluidic channels, and lengths from a boundary of the microfluidic channels formed by the concave portion to the other end of the microfluidic channels are equal to each other.

The chamber includes a sperm collector extending and connected to the other end of the microfluidic channels to collect spermatozoa having forward progression.

In addition, the first chamber cover is provided with an openable discharge port formed at a position corresponding to the sperm collector and configured to be opened or closed as needed.

The chamber includes a feedback channel connected between one side of the sperm collector and one end of the microfluidic channels.

In addition, the feedback channel is formed by two feedback channels.

The chamber includes an air collector connected to one side of the sperm collector to collect air.

In addition, the sperm collector and the air collector are connected by a zigzag-shaped connection channel.

### Advantageous Effects

According to one embodiment of the present invention, there is provided a male infertility diagnosis system including a microfluid chip, wherein the microfluid chip includes a first chamber cover having an injection port allowing a medium and a sperm sample to be injected therethrough, and a chamber coupled to a lower portion of the first chamber cover and provided with a plurality of microfluidic channels, wherein the plurality of microfluidic channels is arranged to converge in a direction in which spermatozoa progress.

The male infertility diagnosis system according to one embodiment of the present invention includes a second chamber cover coupled to a lower portion of the chamber to form a base surface of the microfluidic channels.

The male infertility diagnosis system according to one embodiment of the present invention includes a sperm detector arranged under the second chamber cover to monitor the spermatozoa in the chamber through diffraction of light emitted from a light source.

The microfluidic channels according to one embodiment of the present invention have a width reduced from one end of the microfluidic channels to the other end of the microfluidic channels.

The injection port according to one embodiment of the present invention is opened to cover at least a part of each of the microfluidic channels.

The injection port according to one embodiment of the present invention is provided with a concave portion curved toward one end of the microfluidic channels.

The concave portion according to one embodiment of the present invention is formed to extend over an upper portion of the microfluidic channels, and the lengths from a boundary of the microfluidic channels formed by the concave portion to the other end of the microfluidic channels are equal to each other.

In addition, the chamber according to one embodiment of the present invention includes a sperm collector extending and connected to the other end of the microfluidic channel to collect spermatozoa having forward progression.

The first chamber cover according to one embodiment of the present invention is formed at a position corresponding to the sperm collector, and is provided with an openable discharge port configured to be opened or closed as needed.

The chamber according to one embodiment of the present invention includes a feedback channel connected between one side of the sperm collector to one end of the microfluidic channels.

The feedback channel according to one embodiment of the present invention is formed by two channels.

The chamber according to one embodiment of the present invention includes an air collector connected to one side of the sperm collector to collect air.

In addition, the sperm collector and the air collector according to one embodiment of the present invention are connected by a zigzag-shaped connection channel.

### DESCRIPTION OF DRAWINGS

FIG. 1 is a schematic diagram illustrating the operation principle of a conventional male infertility diagnosis system.

FIG. 2 is an exploded perspective view of a microfluidic chip used in a male infertility diagnosis system according to one embodiment of the present invention.

FIG. 3 is a perspective view of the microfluidic chip used in the male infertility diagnosis system according to one embodiment of the present invention.

FIG. 4 is a plan view of the microfluidic chip used in the male infertility diagnosis system according to one embodiment of the present invention.

FIG. 5 is a plan view of a chamber used in the microfluidic chip according to one embodiment of the present invention.

FIG. 6 is an exploded perspective view of a microfluidic chip used in a male infertility diagnosis system according to another embodiment of the present invention.

FIG. 7 is a perspective view of the microfluidic chip used in the male infertility diagnosis system according to another embodiment of the present invention.

FIG. 8 is a plan view of the microfluidic chip used in the male infertility diagnosis system according to another embodiment of the present invention.

FIG. 9 is a plan view of a chamber provided with a single feedback channel and used in the microfluidic chip according to another embodiment of the present invention.

FIG. 10 is a plan view of a chamber provided with a plurality of feedback channels and used in the microfluidic chip according to another embodiment of the present invention.

FIGS. 11A and 11B are plan views of a microfluidic chip used in a male infertility diagnosis system according to yet another embodiment of the present invention.

### MAIN REFERENCE NUMERALS IN THE DRAWINGS

10: Microfluidic chip
20: Light source
30: Optical filter
40: Protective glass
50: Sperm detector
100: Chamber
110: Microfluidic channel
111: One end of microfluidic channel
112: Other end of microfluidic channel
120: Sperm collector
130: Connection channel
140: Feedback channel
141: Additional feedback channel
150: Air collector
200: First chamber cover
210: Injection port
211: Concave portion
212: Openable discharge port
220: Discharge port
300: Second chamber cover

### BEST MODE

The advantages and/or features of the present invention and the method of achieving the same will be clearly understood from the following detailed description of the embodiments taken in conjunction with the accompanying drawings. It should be understood, however, that the present invention is not limited to the embodiments disclosed herein and may be embodied in many different forms. These embodiments are provided so that this disclosure will be thorough and complete, and will fully convey the scope of the invention to those skilled in the art. The invention is only defined by the appended claims. Like reference numerals refer to like elements throughout the specification.

Hereinafter, embodiments of the present invention will be described in detail with reference to the accompanying drawings.

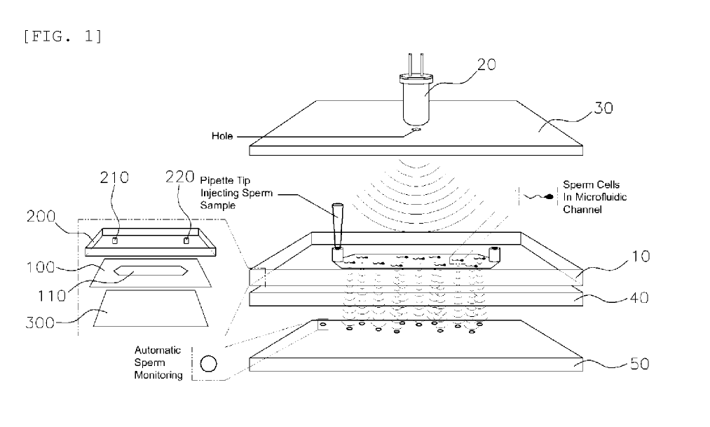

FIG. 1 is a schematic diagram illustrating the operation principle of a conventional male infertility diagnosis system.

Referring to FIG. 1, the conventional male infertility diagnosis system includes an LED corresponding to a light source 20, an optical filter 30 having a hole through which light generated from the LED passes, a microfluidic channel 10 configured to measure movement of spermatozoa and provided with a microfluidic channel 110 allowing a sperm sample to be injected thereinto for movement of spermatozoa in the sperm sample, a protective glass 40 configured to protect the microfluidic chip 10, a charge-coupled device (CCD) sensor corresponding to a sperm detector 50 configured to monitor the spermatozoa through the shadow of the spermatozoa generated by diffraction of light.

Here, the microfluidic chip 10 may be composed of a plurality of layers. The microfluidic chip 10 includes a first chamber cover 200 provided with an injection port 210 for injecting a sperm sample collected from a subject or a medium and a discharge port 220 for extracting the sperm sample after a test, a chamber 100 provided with a microfluidic channel 110 coupled to a lower portion of the first chamber cover 200, and a second chamber cover 300 coupled to a lower portion of the chamber 100 and formed of a transparent material.

As an example, the first chamber cover 200 may be formed of polymethylmethacrylate (PMMA), the chamber 100 may be formed of a double-sided adhesive (DSA), and the second chamber cover 300 may be formed of glass. However, embodiments are not limited to such materials and may include any materials used in the art.

The male infertility diagnostic system may be operated through the following process.

First, a human tubal fluid (HTF) medium containing bovine serum albumin (BSA) is injected into the microfluidic channel 110 of the chamber 100 through the injection port 210 of the first chamber cover 200, using an injection tool operated using pressure.

Then, a thin mineral oil layer is formed in the discharge port 220 of the first chamber cover 200 to prevent evaporation of the medium.

Thereafter, the sperm sample is injected through the injection port 210 of the first chamber cover 200, and then the microfluidic chip 10 is placed on the sperm detector 50. Then, by acquiring a shadow pattern of spermatozoa generated by diffraction of light and monitoring movement of the sperm, male infertility may be diagnosed.

However, the conventional male infertility diagnosis system, which injects the medium or the sperm sample using an injection tool operated using pressure, causes inconvenience as it requires additional devices such as the injection tool. Further, measurement accuracy is lowered as pressure is applied to the inside of the injection port 210 due to the characteristics of the injection tool operated using pressure.

In addition, in the conventional male infertility diagnosis system, as the medium partially evaporates before the discharge port 220 of the first chamber cover 200 is closed, a density difference is produced, and thus the medium moves toward the discharge port 220. As a result, movement of some spermatozoa is induced irrespective of the forward progression of the spermatozoa. Thereby, measurement accuracy is lowered.

In order to address the issues mentioned above, the present invention proposes a male infertility diagnosis system which can be used without the injection tool operated using pressure, and has excellent measurement accuracy as no discharge port is provided. Details of the proposed male infertility diagnosis system are disclosed below.

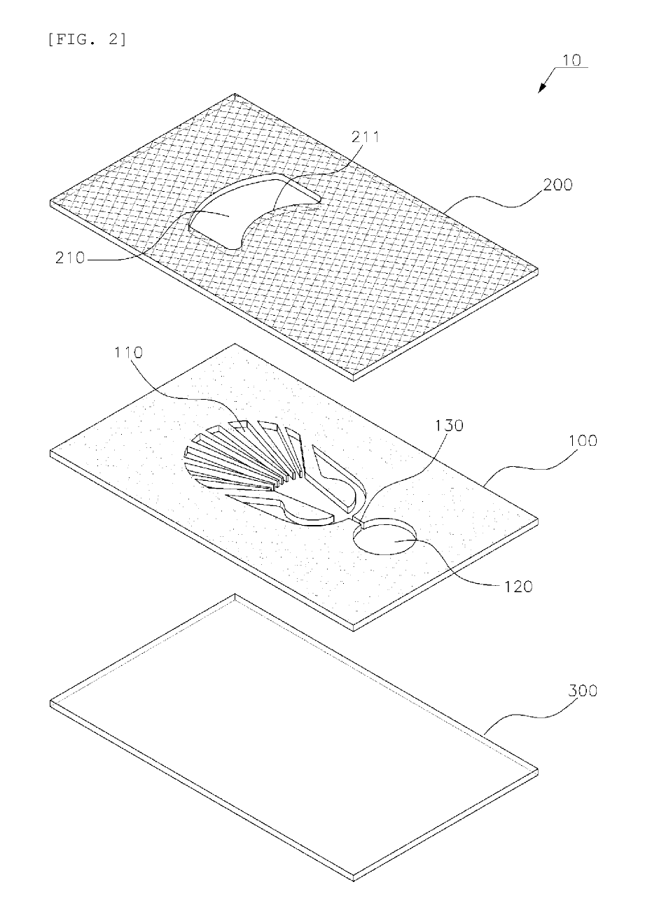

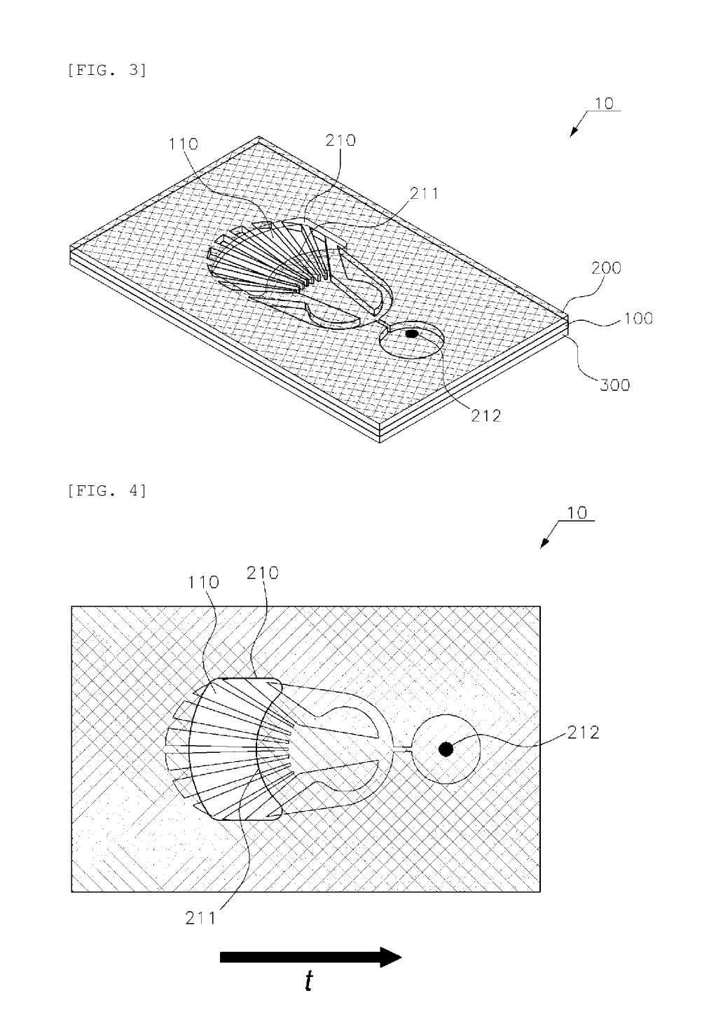

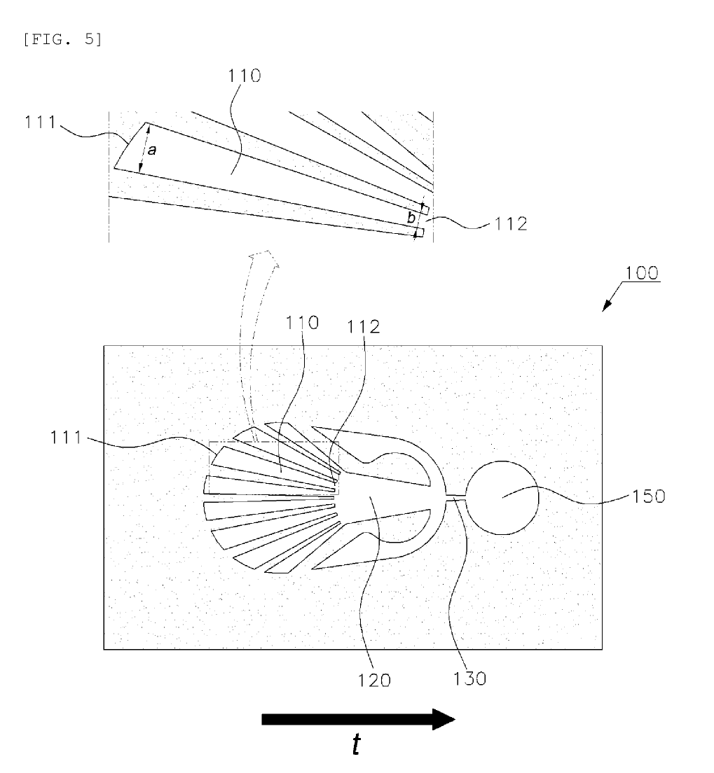

FIG. 2 is an exploded perspective view of a microfluidic chip used in a male infertility diagnosis system according to one embodiment of the present invention, and FIG. 3 is a perspective view of the microfluidic chip used in the male infertility diagnosis system according to one embodiment of the present invention. FIG. 4 is a plan view of the microfluidic chip used in the male infertility diagnosis system according to one embodiment of the present invention, and FIG. 5 is a plan view of a chamber used in the microfluidic chip according to one embodiment of the present invention.

Referring to FIGS. 2 to 5, the male infertility diagnosis system according to one embodiment of the present invention includes a microfluidic chip 10, wherein the microfluidic chip 10 includes a first chamber cover 200, a chamber 100, and a second chamber cover 300.

The first chamber cover 200 is coupled to the top of the chamber 100 and is provided with an injection port 210 through which a medium and a sperm sample are injected. For the first chamber cover, PMMA may be used, but embodiments are not limited thereto.

The medium is a material that mediates movement of spermatozoa in the sperm sample, and may include human tubal fluid (HTF) or various aqueous solutions, saline solutions, and water, which allow smooth movement of the spermatozoa without obstructing movement of the spermatozoa.

Particularly, the first chamber cover 200 according to the present invention is not provided with the discharge port 220. Accordingly, evaporation of the medium injected through the injection port 210 may be minimized, and thus accuracy of measurement of forward progression of the spermatozoa may be enhanced.

The chamber 100 is coupled to the lower portion of the first chamber cover 200 and is provided with a plurality of microfluidic channels 110. The microfluidic channels 110 converge in a direction in which the sperm having forward progression moves. The chamber may be formed of a hydrophilic material or DSA, but embodiments are not limited thereto.

The microfluidic channel 110 may be arranged in a direction in which the spacing between the microfluidic channels 110 is narrowed, such that sperm exhibiting forward progression can be accommodated in one integrated space.

As the microfluidic channel 110 has the above-described structure, one end of the microfluidic channels 110 occupies a relatively large area, and the injection port 210 for covering the vicinity of one end of the microfluidic channels 110 is designed to be large and thus facilitates injection of the sperm sample. The other end of the microfluidic channels 110 may occupy a relatively small area and more easily collect the spermatozoa moving to the other end of the microfluidic channel 110.

Here, one end of the microfluidic channels 110 is located on the side toward which the spermatozoa exhibiting forward progression progress, and the other end of the microfluidic channels 110 is located on the side opposite to the side toward which the spermatozoa exhibiting forward progression progress. Both sides will have the same meaning in the following description.

The second chamber cover 300 is coupled to the lower portion of the chamber 100 and forms a base surface of the microfluidic channels 110. The second chamber cover 300 may be formed of a transparent material, particularly a glass material, but embodiments are not limited thereto. When the second chamber cover 300 is made of a transparent material, light diffracted toward the sperm detector 50 may pass therethrough.

The microfluidic channels 110 are formed to penetrate the chamber 100. Accordingly, in order to enable movement of a fluid through the microfluidic channels 110, an element to close the lower portion of the microfluidic channels 110 like the second chamber cover 300 needs to be separately mounted. The second microfluidic channels 110 may also be integrated with the chamber 100.

The male infertility diagnosis system according to one embodiment of the present invention may include a sperm detector 50 provided under the second chamber cover 300 to monitor the spermatozoa in the chamber 100 by diffraction of light emitted from the light source 20. As the sperm detector, a CCD sensor may be used, but embodiments are not limited thereto.

The width of the microfluidic channel 110 decreases from one end 111 of the microfluidic channel to the other end 112 of the microfluidic channel. The width of the vicinity of one end 111 of the microfluidic channel is denoted by a, and the width of the vicinity of the other end 112 of the microfluidic channel is denoted by b, where a is greater than b.

With the structure of the microfluidic channel 110 described above, when a medium is injected into the injection port 210 using a dropper or a cup without using an injection tool operated using pressure, the microfluidic channel can be automatically filled with the medium by capillary action according to the narrowing structure.

The injection port 210 formed in the first chamber cover 200 may be opened to cover at least a part of each of the microfluidic channels 110 located under the first chamber cover 200 in order to inject the medium and the sperm sample into all of the microfluidic channels 110.

Particularly, at one side of the injection port 210, a concave portion 211 curved toward one end of the microfluidic channel 110 may be formed. The concave portion 211 may be formed to extend over the top of each of the microfluidic channels 110. The lengths from the boundary of the microfluidic channels 110 formed by the concave portion 211 to the other ends of the microfluidic channels 112 are equal to each other.

Due to the shape of the injection port 210 as described above, the deviation of the moving distance of the spermatozoa in the sperm sample injected through the injection port 210 may be minimized, thereby improving measurement accuracy of the forward progression of the spermatozoa.

The other end of the microfluidic channels 112 may be provided with a space for accommodating spermatozoa exhibiting forward progression. The chamber 100 may include a sperm collector 120 extended and connected to the other end of the microfluidic channels 112.

The spermatozoa collected by the sperm collector 120 may be regarded as spermatozoa exhibiting forward progression. By obtaining and analyzing such information, male infertility can be diagnosed.

In addition, the first chamber cover 200 according to the present invention may be provided with an openable discharge port 212 formed at a position corresponding to the air collector 150 and configured to be opened or closed as needed. When the openable discharge port 212 is closed, the same effect as that obtained when the discharge port 220 is not provided may be obtained. When the openable discharge port 212 is opened, the same effect as that obtained when the discharge port 220 is provided may be obtained. Accordingly, the openable discharge port may be selectively used.

For example, at normal times or when the medium or the sperm sample is injected, the openable discharge port 212 may be closed. When the air is removed or the spermatozoa having forward progression is collected using a dropper or the like, the openable discharge port 212 may be opened.

The openable discharge port 212 may include any structures capable of opening and closing the discharge port. For example, the openable discharge port may include any known openable device, such as a rubber stopper and a switch structure.

When the openable discharge port 212 is opened, the spermatozoa near the injection port 210 flow into the sperm collector 120 due to pressure. At this time, not only spermatozoa having forward progression but also unhealthy spermatozoa having non-forward progression are introduced into the sperm collector 120. Accordingly, by checking the number of spermatozoa in the sperm collector 120, the total number of spermatozoa in the sperm sample can be known.

The chamber 100 may include an air collector 150 connected to one side of the sperm collector 120 to collect air.

The air collector 150 may perform a function similar to that of the feedback channel 140, which will be described later. When the medium is introduced through the injection port 210, the air already present in the microfluidic channel 110 even before introduction of the medium moves to the air collector 150, thereby facilitating filling of the sperm collector 120 with the medium.

The sperm collector 120 and the air collector 150 may be connected by a connection channel 130 extending in a linear direction.

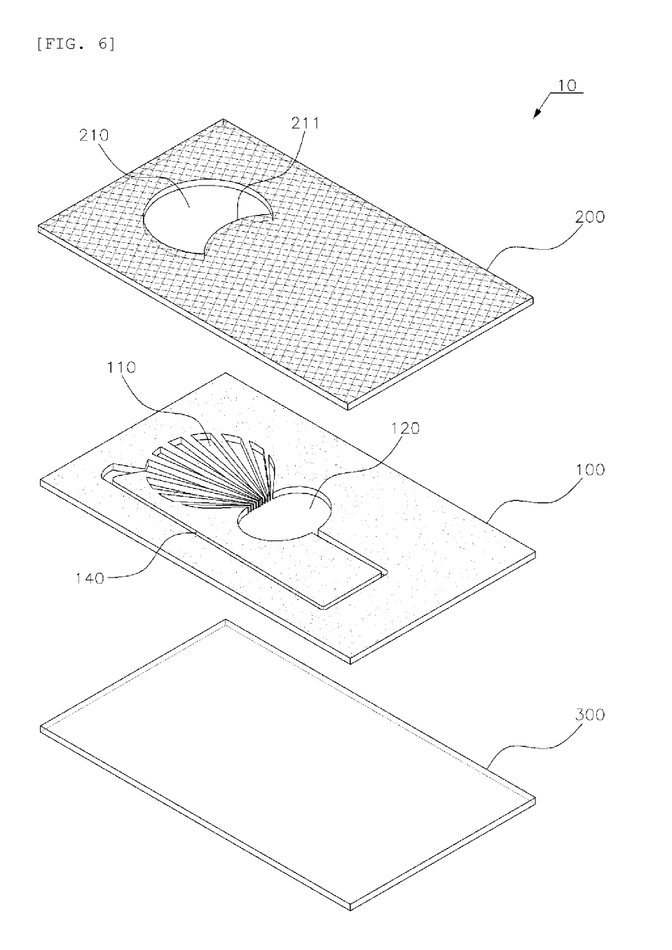

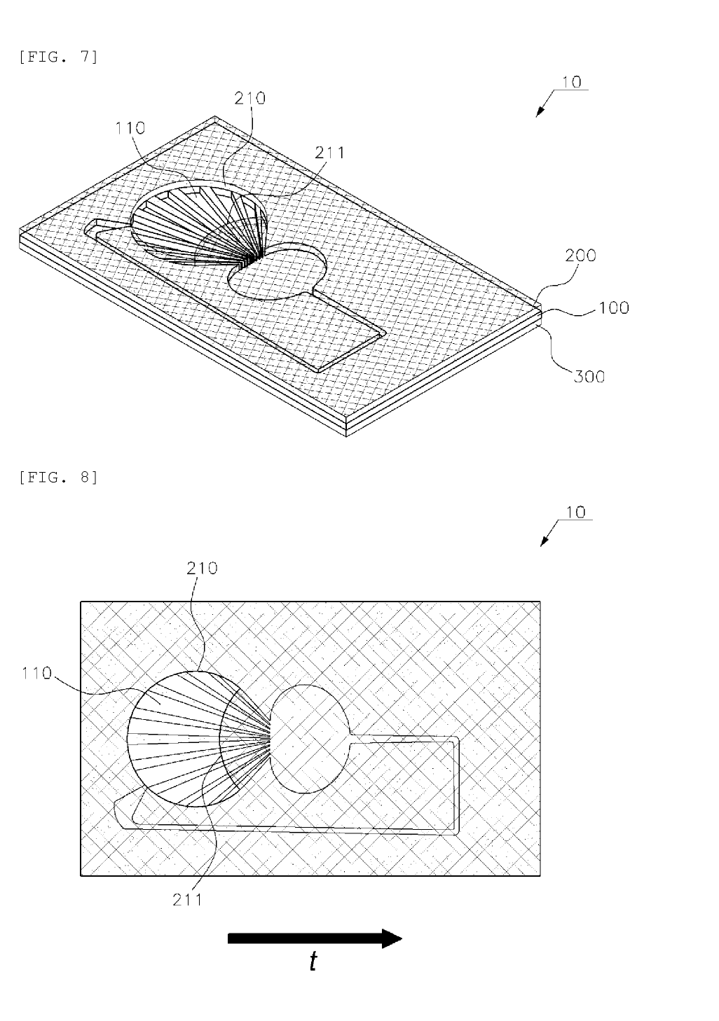

FIG. 6 is an exploded perspective view of a microfluidic chip used in a male infertility diagnosis system according to another embodiment of the present invention, and FIG. 7 is a perspective view of the microfluidic chip used in the male infertility diagnosis system according to another embodiment of the present invention. FIG. 8 is a plan view of the microfluidic chip used in the male infertility diagnosis system according to another embodiment of the present invention, and FIG. 9 is a plan view of a chamber provided with a single feedback channel and used in the microfluidic chip according to another embodiment of the present invention.

Referring to FIGS. 6 to 9, the chamber 100 may include a feedback channel 140 connected between one side of the sperm collector 120 and one end 111 of the microfluidic channel.

The feedback channel 140 according to the present invention is used to remove air and performs a function similar to that of the discharge port 220 formed in the conventional chamber 100. That is, when the medium is injected through the injection port 210, the air already present in the microfluidic channel 110 even before introduction of the medium moves through the feedback channel 140, thereby facilitating filling of the sperm collector 120 with the medium.

In conventional cases, as the medium is evaporated due to the discharge port 220, the accuracy of measurement of forward progression is degraded. The feedback channel 140 according to the present invention prevents evaporation of the medium by replacing the conventional discharge port 220. As a result, the accuracy of measurement of forward progression of spermatozoa is high.

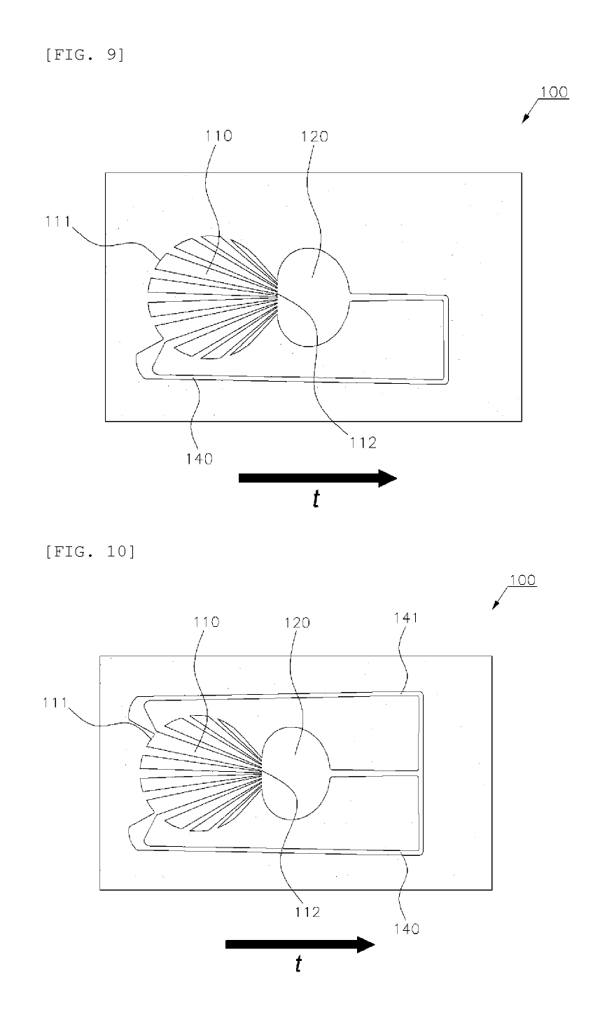

FIG. 10 is a plan view of a chamber provided with a plurality of feedback channels and used in the microfluidic chip according to another embodiment of the present invention.

Referring to FIG. 10, two feedback channels 140 may be formed in order to improve the efficiency of removing air. In this case, in order to efficiently utilize the space, the feedback channel 140 and the additional feedback channel 141 may be formed in the opposite directions.

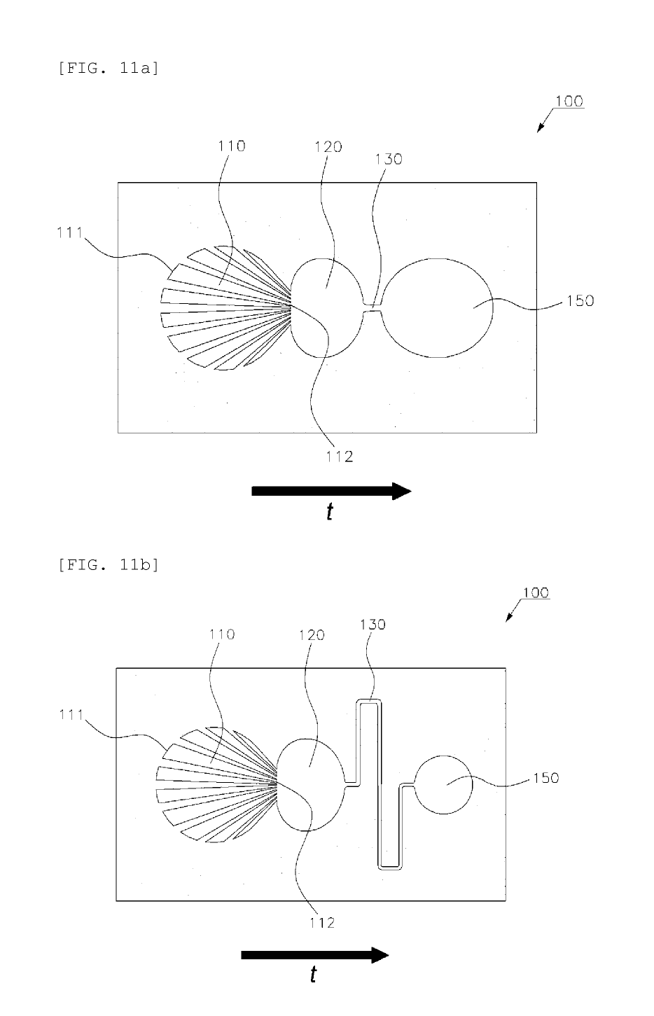

FIGS. 11A and 11B are plan views of a microfluidic chip used in a male infertility diagnosis system according to yet another embodiment of the present invention.

The chamber 100 may include an air collector 150 connected to one side of the sperm collector 120 to collect air.

The air collector 150 may perform a function similar to that of the feedback channel 140. When the medium is injected through the injection port 210, the air already present in the microfluidic channel 110 even before introduction of the medium moves to the air collector 150, thereby facilitating filling of the sperm collector 120 with the medium.

The sperm collector 120 and the air collector 150 may be connected by a connection channel 160 extending in a linear direction, as shown in FIG. 11A. Alternatively, as shown in FIG. 11B, the sperm collector 120 and the air collector 150 may be connected by a zigzag-shaped connection channel 160 to increase a collectible amount of air. Thereby, filling efficiency of the medium may be further improved.

While the invention has been shown and described with reference to specific embodiments thereof, it will be understood by those skilled in the art that various changes and modifications may be made therein without departing from the spirit and scope of the invention. Therefore, the scope of the present invention should not be limited by the described embodiments, but should be determined by the appended claims and equivalents thereof.

While the present invention has been particularly shown and described with reference to exemplary embodiments thereof, and the accompanying drawings, it is to be understood that the invention is not limited to the disclosed exemplary embodiments. It will be understood by those skilled in the art that various modifications and variations can be made in the present invention. Accordingly, it is intended that the scope of the present invention be defined only by the appended claims, and all equivalents or equivalent variations thereof fall within the scope of the present invention.

1. A system for diagnosing male infertility comprising a microfluidic chip,
wherein the microfluidic chip comprises:
a first chamber cover having an injection port allowing a medium and a sperm sample to be injected therethrough; and
a chamber coupled to a lower portion of the first chamber cover and provided with a plurality of microfluidic channels, the plurality of microfluidic channels being arranged to converge in a direction in which spermatozoa progress.

2. The system of claim 1, further comprising a second chamber cover coupled to a lower portion of the chamber to form a base surface of the microfluidic channels.

3. The system of claim 2, further comprising a sperm detector arranged under the second chamber cover to monitor the spermatozoa in the chamber through diffraction of light emitted from a light source.

4. The system of claim 1, wherein the microfluidic channels have a width reduced from one end of the microfluidic channels to the other end of the microfluidic channels.

5. The system of claim 4, wherein the injection port is opened to cover at least a part of each of the microfluidic channels.

6. The system of claim 5, wherein the injection port is provided with a concave portion curved toward one end of the microfluidic channels.

7. The system of claim 6, wherein the concave portion is formed to extend over the microfluidic channels, and lengths from a boundary of the microfluidic channels formed by the concave portion to the other end of the microfluidic channels are equal to each other.

8. The system of claim 4, wherein the chamber comprises a sperm collector extending and connected to the other end of the microfluidic channels to collect spermatozoa having forward progression.

9. The system of claim 8, wherein the first chamber chamber is provided with an openable discharge port formed at a position corresponding to the air collector and configured to be opened or closed as needed.

10. The system of claim 8, wherein the chamber comprises a feedback channel connected between one side of the sperm collector and one end of the microfluidic channels.

11. The system of claim 10, wherein the feedback channel is formed by two feedback channels.

12. The system of claim 8, wherein the chamber comprises an air collector connected to one side of the sperm collector to collect air.

13. The system of claim 12, wherein the sperm collector and the air collector are connected by a zigzag-shaped connection channel.
